(19) United States
(12) Patent Application Publication
(10) Pub. No.: US 2025/0383338 A1
(43) Pub. Date: Dec. 18, 2025

(54) METHOD FOR THE CHARACTERIZATION OF PRODUCTS FOR CELL THERAPY

(71) Applicant: CELLPLY S.R.L., Bologna (IT)

(72) Inventors: Alice BETTELLI, Bologna (IT); Massimo BOCCHI, Bologna (IT); Andrea FAENZA, Bologna (IT); Laura ROCCHI, Bologna (IT)

(21) Appl. No.: 18/879,412

(22) PCT Filed: Jun. 27, 2023
(86) PCT No.: PCT/IB2023/056604

§ 371 Date: Dec. 27, 2024

(30) Foreign Application Priority Data
Jun. 28, 2022 (IT) 102022000013678
Apr. 28, 2023 (IT) 102023000008451

(51) Int. Cl. G01N 33/50; B01L 3/00; G01N 15/10; G01N 15/14; G01N 33/53

(52) U.S. Cl. CPC G01N 33/5014; B01L 3/502715; G01N 15/14; G01N 33/505; G01N 33/53; B01L 2300/0829; B01L 2300/0861; G01N 2015/1006; G01N 2500/10

(57) ABSTRACT

The present invention relates to a method for determining the efficacy of a cell therapy or immunotherapy, where said method comprises; a. Providing a first set of microwells each containing effector cells and target cells; b. optionally, providing a second set of microwells each containing target cells; c. Selecting those microwells comprising a single effector cell and n target cells, where n is between 1 and 50, preferably between 1 and 20; d. Keeping the cells in culture for a time t; e. Measuring the number of dead target cells in each of the microwells selected to contain a single effector cell; f. Calculating a potency score consisting of the weighted average of the number of target cells killed by each effector cell.

### STATE OF THE ART

The assays commonly used to characterize and understand the mode of action of cell therapies, to identify optimal effector cells, to improve the production process in order to maximize the potency of cell therapies by ensuring the minimum exhaustion of the cytotoxic capacity of immune cells or for product release, are cytotoxicity and cytokine release assays revealing immune cell and target cell interactions.

The currently available methods do not allow verifying the functional heterogeneity of cell therapy products, neither establishing whether and how long the cells involved in the target cell killing process maintain a cytotoxic capacity to kill further target cells (known as the serial killing feature). In fact, bulk assays are used, from which a piece of information is derived, given by averaging the cell population under analysis, without the possibility of tracing the cytotoxicity of each individual effector cell or visualizing the progressive activity carried out by each effector cell. The flow cytometry assays, widely used in this context, also define subpopulations only based on phenotypic markers, not based on functionality.

There remains a strong need for a method capable of characterizing a cell therapy product with single-cell-specific information which allows both observing the functional heterogeneity, i.e., how many immune cells exhibit cytotoxic activity against target cells, and assessing how many immune cells are capable of maintaining a cytotoxic activity downstream of a killing activity of one or more target cells, thus qualifying as “serial killer” cells, or instead whether the immune cells exhaust their cytotoxic activity downstream of one or more killing events.

Since several methods are also available to enhance the activity of immune cells against a specific target, there remains a strong need to compare the effectiveness of these different methods in giving some degree of potency and exhaustion to immune cells downstream of the cell therapy modification and production process.

Lastly, considering that the cytotoxicity assays used in cell therapy rely on the ability to maintain target cell viability generally for 4-72 hours, it is fundamental to maintain ideal conditions and minimize the noise in the measurement due to unwanted and un-correlated death of target cells and at the same time ensure that the effector cells are maintained in a local environment where they can exert their cytotoxic activity against target cells, with minimal alteration of the surrounding environment.

Microfluidic devices are available as state-of-the-art tools allowing to carry out large-scale high-content analyses and with single-cell resolution. Working with nano-scale volumes, despite offering unique advantages as the ability to co-localize single effector cells with multiple target cells and measure the cytokines secreted by individual cells or to subsequently run multiple assays on the same cells for extended periods, requires the highest level of control of the local environment to minimize the death of target cells due to factors unrelated to effector-target interactions.

### DESCRIPTION

It forms an object of the present invention a method suitable for cell-mediated cytotoxicity assays.

In an embodiment, the method is carried out in an open microwell microfluidic device, capable to enable an automated rapid liquid exchange into the wells without displacement of the suspension cells loaded. This capability enables to automate assay preparation and execution in the wells on the sample previously loaded and to feed microwells with nutrients at desired time intervals thus achieving a periodic perfusion of media.

### DESCRIPTION OF THE DRAWINGS

FIG. 1: Exemplary diagram of an inverted open microwell system used in the method of the present invention, perspective view (A), vertical section (B) and top view (C).

FIG. 2: Workflow showing (A) delivery of cells into microwells, (B) cell integrated staining, (C) perfusion of channels with culture medium supplemented with Propidium Iodide (PI), (D) incubation with no perfusion. Steps (C) and (D) can be re-iterated to generate periodic perfusions multiple times and at specific time points.

FIG. 3: Cell viability of KG-1 (A) and K562 (B) at 72 h. While for K562 cells data show no statistical difference between the conditions, for KG-1 the 24 h periodic perfusion preserves cell viability while limiting changes to the environment surrounding the cells. (C) Kinetics of KG-1 and K562 cell viability and cell count over time-lapsed 72 h assay. Data refers to the 24 h perfusion condition.

FIG. 4: Image sequence of a KG-1 cell line, showing that cell viability is maintained as well as the persistence of CMAC marker, with an increase in the number of cells over time due to proliferation. PI marker is not visible as no cell death was observed in this microwell.

FIG. 5: Cellular viability measured after 72 hours in culture, measured as the re-perfusion frequency varies. Solid line: target cells only. Dotted line: target cells co-cultured with effector cells.

FIG. 6: (A) representative image of a set of microwells visualized by fluorescence and phase-contrast microscopy. The arrows indicate the effector cell, highlighted with Calcein AM staining and the marker CD16/CD56. The wells containing a single effector cell, circled in the figure, are selected for analysis, the results of which are shown in the graph. (B) graph showing the % of effector cells which are capable of killing the indicated number of target cells.

FIG. 7: representative image of a set of microwells containing (A) an inactive effector cell or (B) an active effector cell. The effector cell is highlighted by the arrow.

FIG. 8: quantization of potency of the effector cells obtained from donors D1-D6. (A) effector cells killing 1 or 2 target cells; (B) effector cells 3 or more target cells; (C) effector cell potency derived from the results of graphs (A) and (B).

FIG. 9: potency of the effector cells from donors D1-D3.

FIG. 10: potency on two different phenotypes of the effector cells (A) obtained from donor DA; (B) obtained from donor DC.

### DETAILED DESCRIPTION

### Definitions

By interference coupling it is meant herein a cooperation between two elements, so that said two elements can be considered as joined. When said two elements, in this case a tip and a vertical channel, are coupled by interference, a fluid charged into said tip and released in said vertical channel is forced to move within the channel, said interference coupling being such as to prevent the passage of fluid, i.e., said interference coupling is such as to mutually seal the two elements.

By connector it is meant herein any tubular, cylindrical, more or less tapered, converging or diverging element adapted to put two compartments in fluidic connection.

By re-perfusion it is meant herein the replacement of the medium in which the culture is present with fresh medium. Said fresh medium is either the same as or is different from the medium in which said culture is already present. In an embodiment, said medium is a culture medium. In an embodiment, it is a culture medium comprising one or more drugs and/or one or more dyes, and/or one or more labeled antibodies and/or one or more cell viability markers.

Fluids: any substance in liquid or gas form.

Biological sample: sample comprising cells obtained from a micro-organism, an animal and/or a human, preferably a human, where said sample is preferably selected from the group comprising biological fluids or biopsies. Said sample comprises suspended cells or it is a tissue. In a preferred embodiment, it is a sample of blood or a bone marrow aspirate. Alternatively, said biological sample consists of cultured cells, such as a cell line, or a composition comprising cultured cells and cells from a patient.

High-content assay: phenotypic assay conducted on cells.

Time-lapse: imaging technique involving a series of shots of the same field taken in a time sequence.

Ex-vivo: testing performed on a tissue obtained from an organism into an environment outside the organism itself, with minimal alteration of natural conditions.

By long term it is meant a culture maintained over 24 hours, or 48 hours, preferably 72 hours.

Serial killer cells: immune cells capable of producing each a cytotoxic activity on two or more target cells, thus causing their death.

In an embodiment, the method is performed in the open microwell microfluidic device described in WO2017/216739.

In an embodiment, the open microwells are about 0.5 nL open microwell.

Advantageously, the open microwell interface provides a source of gas exchange which contributes to long term viability.

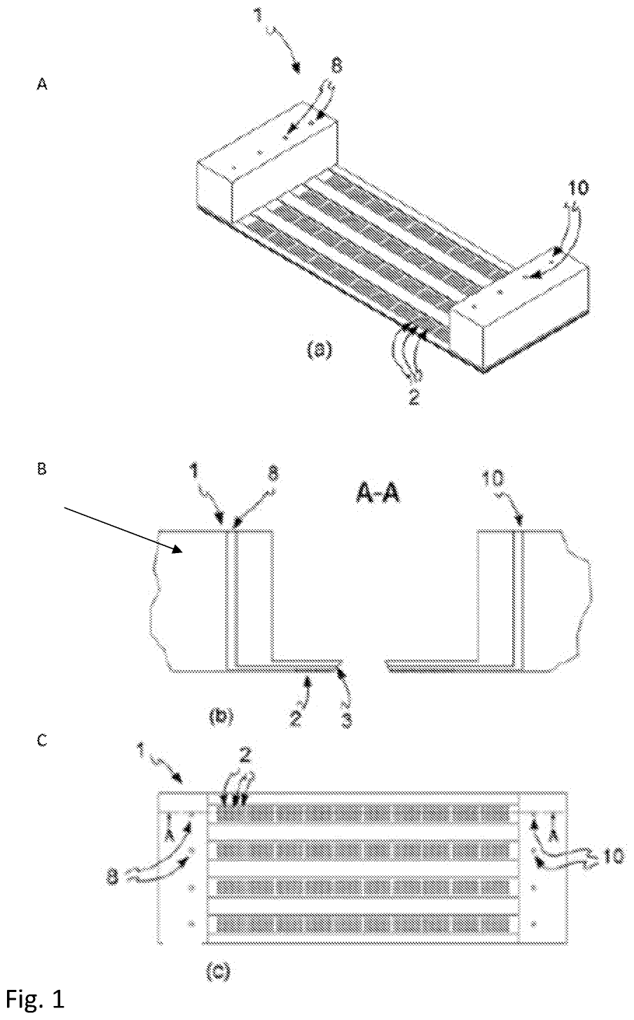

In an embodiment, the here claimed method is performed by providing a kit which comprises a tip, and a microfluidic device (1), FIG. 1, which comprises at least one microchannel (3) and an input region (8) which comprises at least one vertical channel (18), said tip and said vertical channel (18) being dimensioned so to produce an interference coupling therebetween.

Said tip is selected from one of the tips commercially available which comprise at least one proximal portion intended to cooperate with a fluid dispensing system and an open tapered distal portion.

Preferably, said distal portion of said tip and said vertical channel (18) are made of plastic and make the system resilient enough to ensure the seal, avoiding gaskets. In a particularly preferred embodiment, the system geometries described hereinafter ensure that the contact between said vertical channel (18) and said tip does not occur in a single point but is distributed on a surface portion, further ensuring an effective seal. This condition is advantageously verified where the semi-opening angle of said terminal portion of said tip and said vertical channel (18) are little different, preferably differ by less than 10°. Even more preferably, said vertical channel (18) is a cylinder, optionally slightly tapered downwards.

The method of loading/unloading fluids in the microfluidic device (1) comprised in the kit described comprises the following steps:
a) Providing a kit according to the present description;
b) Optionally, charging a fluid into said tip;
c) Positioning said tip above said input region (8) and inserting it up to reaching an interference coupling position between said distal region of said tip and said vertical channel (18) in said input region (8);
d) Releasing the fluid contained in said tip in said microfluidic device (1) through said input region (8) or, alternatively, with the same tip, suctioning fluid already contained in said microfluidic device.

A microfluidic device is also described, which is an inverted open microwell system which comprises an array of open microwells (2), at least one microchannel (3), at least one input port (8) for reagents and/or for one or more biological samples and at least one output port (10) for them, said input and output ports being in microfluidic communication with one or more of said microchannels (3), wherein said microchannel (3) has a cross-section area of micrometric dimensions and provides fluid to said microwells (2), wherein said inverted open microwell system is, in one embodiment, inserted in an automated management system which comprises the following features: an incubator at controlled temperature, humidity and CO₂, fluid dispensing system, phase-contrast and fluorescence image acquisition.

Said automated management system is achieved by assembling elements which are known in the art as a temperature, humidity and CO₂ control incubator, microplate pipetting systems, fluorescence and phase-contrast microscopy lenses connected to an image acquisition camera, such as a CMOS or CCD camera, where said elements are managed in whole or in part by software known to those skilled in the art through hardware connected thereto.

In a particularly preferred embodiment, each microchannel (3) is associated with an input port (8) and an output port (10).

In a preferred embodiment, the microfluidic device (1) also comprises reservoirs, where said reservoirs are at least one reservoir for reagents and at least one reservoir for one or more biological samples. Said reservoirs are selected from the group comprising: plates, one or more multiwell plates, such as 96-well plate, Eppendorf tubes. Said reservoirs may be 2, or 4, 8, 16, 24, 48, 96, 384.

In an embodiment, the method according to the present invention comprises:
Making available at least two cellular population, wherein at least a first cellular population comprises effector cells growing in suspension, at least a second cellular population comprises target cells;
Co-culturing said cells for at least 24 h, for example 48h or 72 h, periodically re-perfusing the culture, wherein said re-perfusion periodically occurs with a time range comprised between 1 and 30 hours, for example once every two hours, or once every 12 hours, or once every 24 hours;
Evaluating cell viability.

In an embodiment, said method is carried out in a microfluidic system.

In an embodiment, said method is carried out in an inverted open microwell system (1) which comprises an array of open microwells (2), at least one microchannel (3), at least one input port (8) for reagents and/or for one or more biological samples and at least one output port (10) for them, said input and output ports being in microfluidic communication with one or more of said microchannels (3), wherein said microchannel (3) has a cross-section area of micrometric dimensions and provides fluid to said microwells (2).

In an embodiment, said microfluidic device comprises 16 microchannels (3). In an embodiment, 1,200 open microwells (2) are connected to each of said microchannels (3).

In an embodiment, said inverted open microwell system is operated by an automated management system which comprises the following features: incubator at controlled temperature, humidity and CO₂, fluid dispensing system, phase-contrast and fluorescence image acquisition.

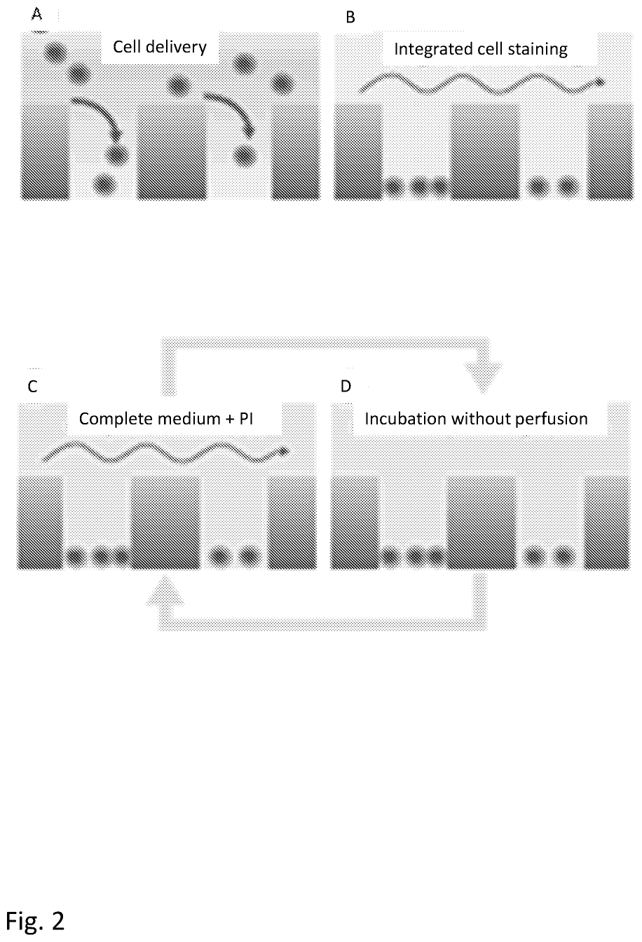

In an embodiment, said method comprises:
Providing an inverted open microwell system (1) which comprises an array of open microwells (2), at least one microchannel (3), at least one input port (8) for reagents and/or for one or more biological samples and at least one output port (10) for them, said input and output ports being in microfluidic communication with one or more of said microchannels (3), wherein said microchannel (3) has a cross-section area of micrometric dimensions and provides fluid to said microwells (2);
Providing an automated management system of said inverted open microwell system which comprises the following features: incubator at controlled temperature, humidity and CO₂, fluid dispensing system, phase-contrast and fluorescence image acquisition.
Placing said inverted open microwell system (1) in said automated system;
Charging reagents through one or more of said input ports (8), wherein said reagents comprise: filling buffer and/or washing solution and/or one or more drugs and/or one or more dyes, and/or one or more labeled antibodies and or one or more cell viability markers;
Charging said at least two cellular population,
Staining said cells with one or more dyes and/or one or more labeled antibodies and or one or more cell viability markers (FIG. 2B);
Acquiring images from one or more of said microwells (2), at a timepoint TO;
Periodically re-perfunding the culture with fresh media, optionally comprising one or more drugs and/or one or more dyes, and/or one or more labeled antibodies and or one or more cell viability markers (FIG. 2C-D);
Acquiring images from one or more of said microwells (2), at a timepoint T1;
   wherein said steps of charging and acquiring images are reiterated over time. In an embodiment, said at least two cellular population are a population of effector cells charged at a concentration of about 1.2×10⁵ cells/mL and a population of target cells charged at a concentration of 3.3×10⁶ cells/mL (FIG. 2A).

In an embodiment, one of said at least two cell populations is an effector cell population.

In an embodiment, a single effector cell is loaded in at least 400 of the 1,200 microwells (2) connected to each microchannel (3).

In an embodiment, one of said at least two cellular population are KG-1, a human macrophages cell line.

In an embodiment, one of said at least two cellular population are K562, a human lymphoblasts cell lines.

In an embodiment, said effector cells are NK lymphocytes.

In an embodiment, said effector cells are T lymphocytes.

In an embodiment, said T or NK cells are genetically modified.

Said effector cells are co-cultured in the presence of target cells.

Said target cells are primary cells obtained from a subject, or they are a cell line.

In an embodiment, said target cells express the antigen recognised by said effector cells.

In an embodiment, the assay and the time lapse are performed for up to 72 h.

In an embodiment, said marker is used for cell detection, and it is 7-amino-4-chloromethylcoumarin (CellTracker™ Blue CMAC).

In an embodiment, said marker is used to assess cell death, and it is Propidium Iodide (PI).

In an embodiment, said marker is Calcein AM.

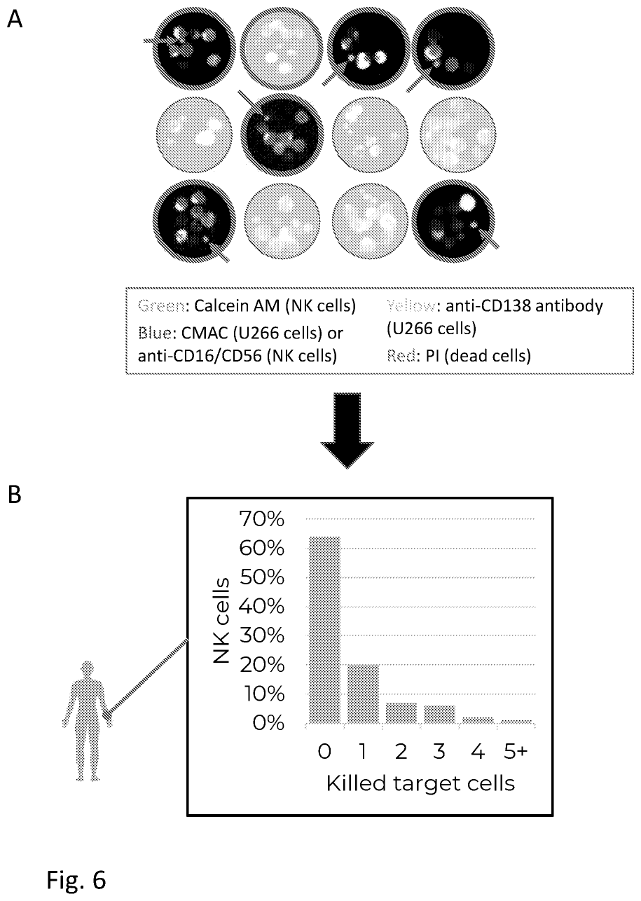

In an embodiment, homogeneous co-cultures are selected, i.e., those microwells containing a single effector cell and target cells in a number within a defined range are selected. By way of example, with reference to FIG. 6A, microwells containing a single effector cell NK, highlighted by Calcein AM staining, are selected. In said microwells, PI-stained dead target cells are counted. This allows defining the % of effector cells capable of killing 0 target cells, 1, 2, 3, 4 or over 5 target cells (FIG. 6B). From this profile, the so-called potency score is calculated, consisting of the weighted average of the number of target cells killed by each effector cell, having first subtracted from the count the number of target cells dead on average in microwells which do not contain any effector cells.

$$Potency Score = \frac{{\sum }_{i = 1}^{N} = {tk}_{i}}{N}$$

where tkᵢ denotes the number of target cells killed by effector cell i, normalized to the spontaneous death of the same target cells in the absence of effector cells, and N denotes the total number of effector cells.

Therefore, the present invention further relates to a method for determining the efficacy of a cell therapy (or immunotherapy), where said method comprises;
a. Providing a first set of microwells each containing effector cells and target cells;
b. Providing a second set of microwells each containing target cells;
c. Selecting those microwells comprising a single effector cell and n target cells, where n is between 1 and 50, preferably between 1 and 20;
d. Keeping the cells in culture for a time t;
e. Measuring the number of dead target cells in each of the microwells selected to contain a single effector cell;
f. Calculating a potency score consisting of the weighted average of the number of target cells killed by each effector cell, having first subtracted from the average number of target cells dead in the second set of microwells.

In an embodiment, in said step c. microwells containing a number of target cells between 5 and 15, or between 7 and 10, or between 10 and 15, are selected. Advantageously, the selection of microwells comprising a number of target cells within a narrow range leads to more homogeneous co-cultures. Moreover, having microwells comprising at least 5, or at least 7 target cells allows better visualizing the serial killing activity.

In an embodiment, said microwells are inverted open microwells of a microfluidic device as described in the above paragraphs.

In an embodiment, the effector cells are selected only if positive, or only if negative, for a surface marker which is selected, by way of example, from the group comprising CD3, CD4, CD16, CD56, CD45RA, CCR7, NKp30, NKp44, NKp46, PD-1, LAG-3, TIM-3.

In an embodiment, the dead target cells are counted only if positive, or only if negative, for a surface marker which is selected, by way of example, from the group comprising CD38, HLA-DR, CD34.

Said potency score advantageously allows representing the efficacy of the therapy in an absolute manner.

The potency score defined herein is the average number of target cells that a single effector cell is capable of eliminating, having tested this activity by means of a well-controlled replication of the test conditions, i.e., a known number of cells in a known volume.

An advantage of this approach is to allow the comparison between different variants of a cell therapy, e.g., different sources of immune cells (donors or patients), different engineering methods, different production processes, and different processes and approaches for selecting certain subpopulations.

From the data we have so far, it is observed, for example, that a CAR-NK therapy can achieve a score >1 for t=12 h or t=24. In another set of experiments, it is observed that un-engineered NK cells can achieve a score typically <1.

Advantageously, the method according to the present invention allows a combined functional and phenotypic characterization.

For example, having the ability to observe 4 different colors, up to 2 surface markers can be assessed in combination with cytotoxicity. In an embodiment, the cytoplasm of the target cell is stained with CMAC (in the blue DAPI channel), and PI (in the yellow TRITC channel) is used as a cell death marker. It is thus possible to use two surface markers, in the green FITC channel and the red CY5 channel. In this embodiment, for example, it is possible to select the antibody 1-positive cell population, and in the latter to identify serial killer, killer, and inactive cells. This allowed observing that the presence of a surface marker has a correlation with an increase in the potency score (in other words, there are subpopulations defined based on immunophenotype which have greater potency scores than the average potency score of the parent population).

According to the method here described, the viability of target cells can be maintained for 72 h outside of the presence of immune effector cells even in the prolonged presence of specific stains used for cell detection, such as 7-amino-4-chloromethylcoumarin (CellTracker™ Blue CMAC), and time-lapse death assessment, e.g., Propidium Iodide (PI).

The entire setup has been optimized in terms of imaging, fluidics, and staining methods to achieve a reagent concentration which is high enough to let cells to be properly detected and classified, while not significantly impacting on their viability.

The method here described allows to apply these stains for the entire duration of the assay to support a time lapsed analysis.

In conclusion, the conditions here identified ensure a model consisting of target cell lines, a set of stains and cell processing methods that guarantees a downstream use for potency assays based on cell killing analysis.

### EXAMPLES

### Example 1: Viability Analysis Carried Out on KG-1, a Human Macrophages Cell Line, and K562, a Human Lymphoblasts Cell Lines, Loaded, Processed, and Imaged in Time Lapse for Up to 72 h

K562 and KG-1 cell lines were loaded separately in multiple microchannels of a microdevice and the steps of the experimental workflow included:
cell delivery into microwells at a concentration of 2×10⁶ cells/mL;
automated preparation/dilution of cytoplasm marker, CMAC, at 7 μM concentration;
automated cell staining through infusion of CMAC into microfluidic channels
   automated immunophenotyping, with automated reagents dilution, preparation and infusion;
   automated preparation and dilution of cell death marker, PI, at 5 mM concentration with complete medium (RPMI+10% FBS+1% L-Glu+1% Penstrep);
automated cell staining through infusion of complete medium supplemented with PI;
time-lapsed imaging in brightfield/phase-contrast and 4 fluorescent channels repeated every 12 h for 72 h.

Preparation and periodic perfusion of culture media with PI was repeated at determined time-points to freshen up nutrients as well as to restore the fluorescence intensity of PI signal. For each re-infusion 100 μL were injected to each microchannel at a 10 μl/s flow rate (rinsing time: 10 seconds). Test repeatability was assessed replicating the test with independent trials on three different days. Cell viability was measured in time lapse through AI-based single-cell detection and quantification of the percentage of cells with PI uptake.

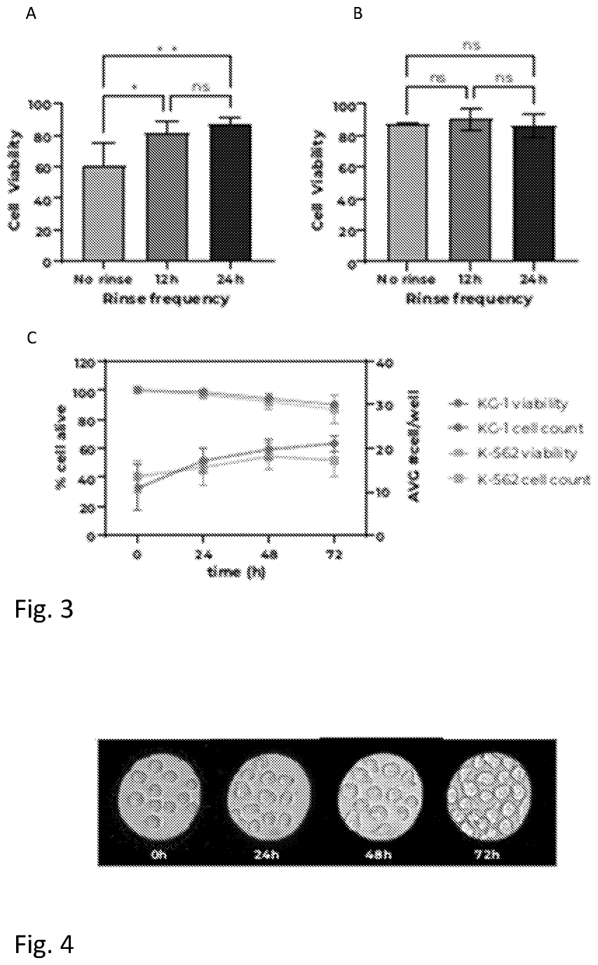

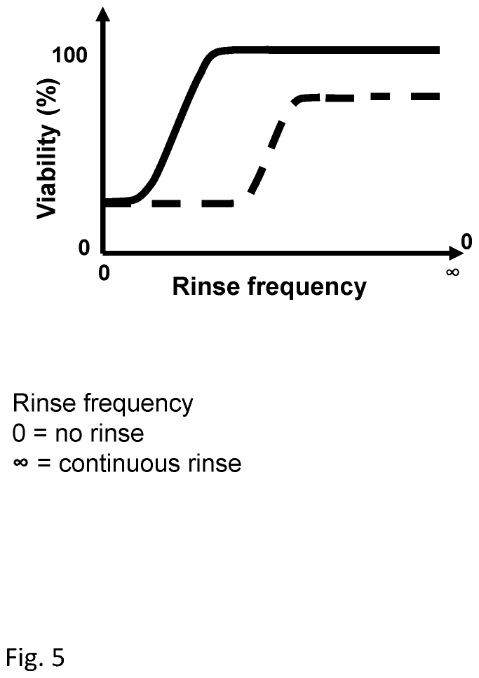

K562 cell line showed an average 72 h end-point viability, after normalization to T0, of 90.58%+6.8% for a perfusion every 12 h, 86.24%+7.3% for a perfusion every 24 h and 87%+0.44% for the no-rinse condition. There was no significant difference between the conditions (FIG. 3B). Results for KG-1 cell line, FIG. 3A, with culture medium perfusion every 12 h or every 24 h showed an average 72 h end-point viability, after normalization to TO, of respectively 80.96%+8.19% and 87.54%+3.36%. For the same cell line, viability at 24 h without media perfusion resulted in 96.71%+2.36% demonstrating an optimal health status. However, cell viability decreased over time until reaching a viability of 61.11%+14.16% at 72 h thus proving the significant advantage brought by a periodic medium perfusion.

Among the two perfusion strategies we tested, there was no statistical difference, so the preferred approach is to rinse every 24 h to minimize the changes on cell surroundings.

By comparison, other micro technologies (Halldorsson et al., Biosensors and Bioelectronics, 2015, 63:218-231) commonly require a continuous perfusion to maintain ideal conditions while the method according to the present invention preserves cell viability by applying short and fast periodic perfusions. As reported in FIG. 3C, these conditions also support cell growth inside the microwells and, as visible in FIG. 4, the CMAC cell tracker propagates to daughter cells and remains visible over the 72 h period.

In FIG. 4, the results of a co-culture experiment are reported, compared to a single culture experiment.

Target cells were co-cultured with effector cells (according to the invention) or per se'.

Cellular viability was assessed after 72 hours of incubation.

Different microwells were exposed to a different re-perfusion frequency, wherein said frequency varied between once in an hour to once in 30 hours. The solid line in the graph represents the target cells in the absence of effector cells. The dotted line, target cells co-cultured with effector cells.

In the absence of any re-perfusion, high % of cell death is observed in the two experimental setting, showing the need to change media to keep an healthy cellular population for up to 72 h.

Increasing the re-perfusion frequency, cells are in good health. At the extreme, the re-perfusion makes the effect of the co-culture moot.

Surprisingly, it is here demonstrated a “working area”, i.e., a re-perfusion frequency optimal to maximise the possibility to measure the effect of the effector cells on the target cells, without having the same impacted by sub-optimal culture condition. Said “working area” is the frequency wherein the maximum delta between the two experimental setting is observed.

Effector cells cultivated per se or in the presence of the target cells show a trend of their vitality comparable to that observed when the target cells are grown alone.

### Example 2: Characterization of 6 Batches for Cell Therapy, Batches D1-D6

Three microfluidic devices, I, II and III, respectively, were provided, each comprising 16 microchannels. Each of said microfluidic devices was loaded with effector cells obtained from donors, said cells being CAR-NK. Specifically, 7 microchannels of each device were loaded with effector cells from one donor, 7 with effector cells from a different donor. 6 of said channels loaded with effector cells were also loaded with target cells, while the seventh was kept as a control channel containing only effector cells. The target cells used were Raji cells, a cell line obtained from a human lymphoma. The remaining 2 channels on each device were loaded with target cells only, as a negative control.

In the co-culture microwells, the effector cells were loaded in a 1:10 ratio with respect to the target cells. For cell tracking, the target cells were stained with CMAC marker. The effector cells were instead visualized in bright field, after assessing that the markers CMAC and Calcein AM, which do not impact the cell viability in the Raji cells, instead induced a proportion of cell death in the CAR-NKs.

By means of an automated process, the effector cells contained in each well were counted, thus resulting in the selection of a total of 1079 wells containing a single effector cell each. Said 1079 microwells were selected for the next steps. The target cells contained in the same wells were then counted.

Note that said results were achieved by loading, for each micro-well, 30 μl of medium where CAR-NK cells were in suspension at a concentration of 1.2*10⁵ cells/ml, then by loading 30 μl of medium where the Raji cells were in suspension at a concentration of 3*10⁶ cells/ml. In the control cases, for each microchannel, there were seeded only 30 μl of medium where CAR-NK cells were in suspension at a concentration of 1.2*10⁵ cells/ml (control of CAR-NK cell viability) or only 30 μl of medium where Raji cells were in suspension at a concentration of 3*10⁶ cells/ml (negative control).

By means of PI staining, the cell death after 24 hours of incubation of the co-culture was then measured.

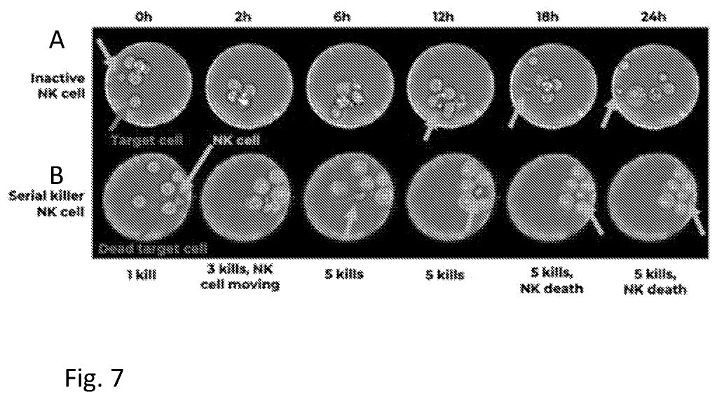

For indicative purposes, images of some microwells are shown in FIG. 7. In panel A, microwells are shown where the CAR-NK cell present is not active, as highlighted by the absence of dead target cells therein. Conversely, panel B shows microwells exemplifying the presence of serial killer CAR-NK cells, where, from left to right, we notice from 1 dead target cell per well to more than 5.

Advantageously, the automated rapid fluid exchange system in the wells allows PI re-marking every 12 hours, so as to compensate for the natural marking loss.

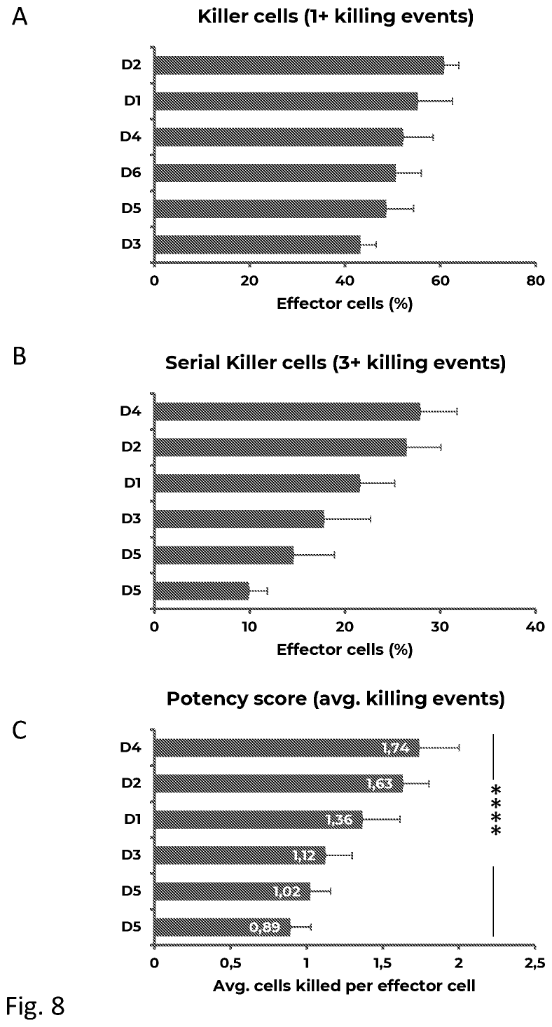

The obtained results were plotted in a graph, shown in FIG. 8. For each donor, the number of NK cells killing 1 or 2 target cells (A), and the number of NK cells killing 3 or more target cells (B) are shown. Finally, panel C shows, for each donor, the potency score, defined according to the present invention. The potency score identifies donor D4 as the most potent, being the one with the highest potency score.

### Example 3: Characterization of 3 Batches for Allogeneic Cell Therapy, Batches D1-D3

NK cells from 3 different donors and target cells were loaded onto the same 16-channel microfluidic device. The target cells used were the K562 cell line. After 24 h of co-culture incubation, the % of NK cells capable of killing 1 target cell, referred to as killer NK cells, and the % of NK cells capable of killing 2 or more target cells, referred to as serial killer NK cells, were assessed.

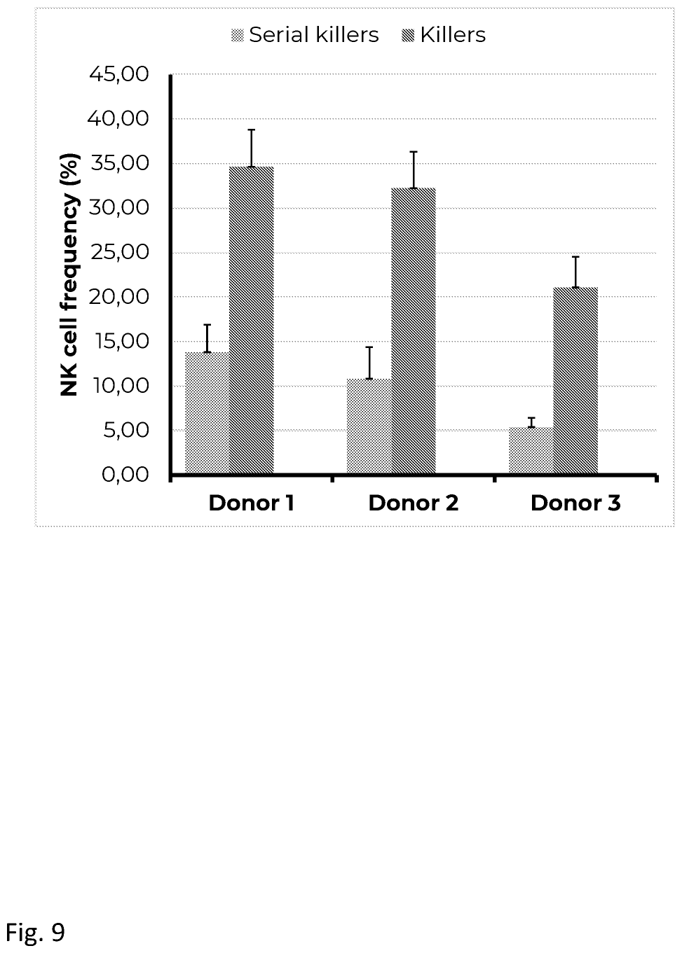

The obtained results are shown in FIG. 9 and show the higher potency of donor D1 as compared to the other two donors tested in the same assay.

### Example 4: Multi-Parametric Phenotypic/Functional Analysis

NK cells from donor A and donor C were provided and plated in a microfluidic device as described in the preceding examples.

Said population of NK cells was co-cultured with a heterogeneous population of target cells, expressing either phenotype A or phenotype B. The potency of the effector cells was then calculated according to the method of the present invention.

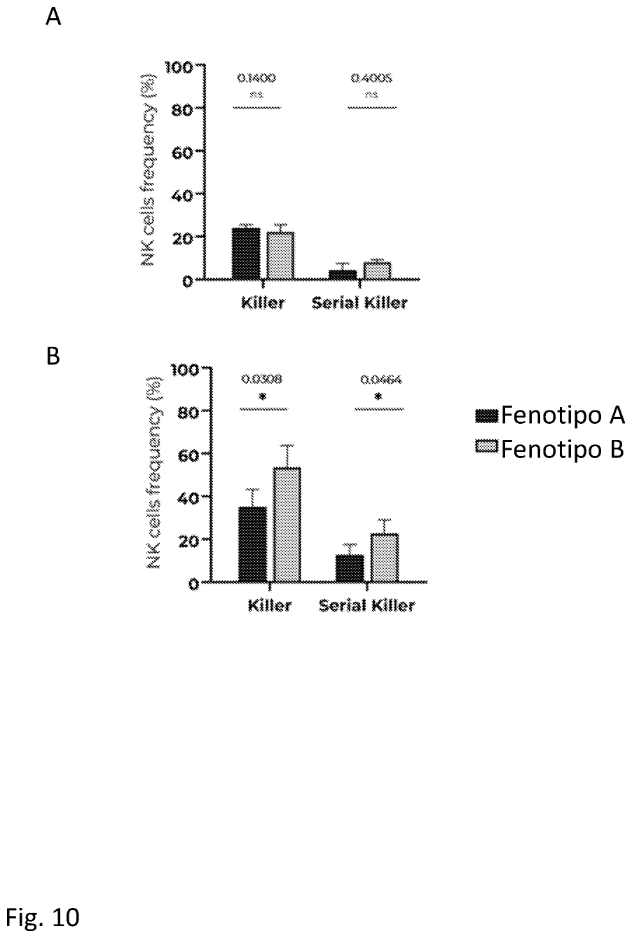

As shown in FIG. 10, for donor C, panel B, an increase in killing potency on cells having phenotype B is apparent.

1. A method suitable for determining the efficacy of a cell therapy or immunotherapy, said method comprising:
a. Providing a first set of microwells each containing effector cells and target cells;
b Optionally, providing a second set of microwells each containing target cells;
c. Selecting those microwells comprising a single effector cell and n target cells, where n is between 1 and 50;
d. Keeping the cells in culture for a time t;
e. Measuring the number of dead target cells in each of the microwells selected to contain a single effector cell;
f. Calculating a potency score consisting of the weighted average of the number of target cells killed by each effector cell.

2. The method according to claim 1, wherein n is comprised between 5 and 15, or between 7 and 10, or between 10 and 15.

3. The method according to claim 1, wherein to calculate said potency score the average of the number of target cells killed in the second set of microwells is subtracted from the average number of target cells killed by each effector cell.

4. The method according to claim 1, wherein said t is comprised from 0 to 120 h, or about 72 h or about 24 h.

5. The method according to claim 1, wherein said method is carried out in a microfluidic system, wherein said method comprises:
Providing an inverted open microwell system which comprises an array of open microwells, at least one microchannel, at least one input port for reagents and/or for one or more biological samples and at least one output port for them, said input and output ports being in microfluidic communication with one or more of said microchannels, wherein said microchannel has a cross-section area of micrometric dimensions and provides fluid to said microwells;
Providing an automated management system of said inverted open microwell system which comprises the following features: incubator at controlled temperature, humidity and CO₂, fluid dispensing system, phase-contrast and fluorescence image acquisition;
Placing said inverted open microwell system in said automated system;
Charging reagents through one or more of said input ports, wherein said reagents comprise: filling buffer and/or washing solution and/or one or more drugs and/or one or more dyes, and/or one or more labeled antibodies and/or one or more cell viability markers;
Charging said at least two cellular populations;
Staining said cells with one or more dyes and/or one or more labeled antibodies and/or one or more cell viability markers;
Periodically re-perfunding the culture with fresh media, comprising one or more drugs and/or one or more dyes, and/or one or more labeled antibodies and/or one or more cell viability markers.

6. The method according to claim 1, wherein into each microchannel are seeded 30 microliter of media wherein effector cells are suspended at a concentration of 1.2×10⁵ cells/mL and 30 microliter of media wherein target cells are suspended at a concentration of 3*10⁶ cells/ml.

7. The method according to claim 1, wherein said effector cells are NK lymphocytes and/or T lymphocytes.

8. The method according to claim 1, wherein said cells are cultured for at least 24 h, periodically re-perfusing the culture, wherein said re-perfusion occurs once in a period of time ranging from 1 to 30 hours.

9. The method according to claim 5, wherein said microfluidic system comprises 16 microchannels.

10. The method according to claim 5, wherein said microfluidic system comprises 1,200 open microwells.

11. A method suitable for long term cell-mediated cytotoxicity assays, wherein said method comprises:
making available at least two cellular populations, wherein at least a first cellular population comprises effector cells growing in suspension, at least a second cellular population comprises target cells;
co-culturing said cells for at least 24 h, periodically re-perfusing the culture, wherein said re-perfusion occurs once in a period of time comprised between 1 and 30 hours;
evaluating cell viability.

12. The method according to claim 11, wherein said co-culture is for at least 72 hours and said re-perfusion occurs once in a period of time comprised between 10 and 26 hours.

13. The method according to claim 11, in which said method is carried out in a microfluidic system.

14. The method according to claim 13, wherein said method comprises:
Providing an inverted open microwell system comprising an array of open microwells, at least one microchannel, at least one inlet port for reagents and/or for one or more biological samples, and at least one outlet port for the same, said inlet and outlet ports being in microfluidic communication with one or more of said microchannels, wherein said microchannel furnishes fluid to said microwells;
Providing an automated management system of said inverted open microwell system that comprises the following features: temperature-, humidity-, and CO₂-controlled incubator, fluid delivery system, phase-contrast and fluorescence imaging;
Placing said inverted open microwell system in said automated system;
Loading reagents through one or more of said inlet ports, wherein said reagents include: filling buffer and/or wash solution and/or one or more drugs and/or one or more dyes, and/or one or more labelled antibodies and/or one or more cell viability markers;
loading said at least two cell populations;
staining said cells with one or more dyes and/or one or more labelled antibodies and/or one or more cell viability markers;
re-perfusing the culture periodically with fresh media, eventually comprising one or more drugs and/or one or more dyes, and/or one or more labelled antibodies and/or one or more cell viability markers.

15. The method according to claim 14, further comprising:
acquiring images from one or more said microwells, at a timepoint TO;
acquiring images from one or more of said microwells, at a timepoint T1;
wherein said steps of charging and acquiring images are reiterated over time.

16. The method according to claim 11, wherein at least two cellular populations are a population of effector cells charged at a concentration of 1.2*10⁵ cells/ml and a population of target cells charged at a concentration of 3.3*10⁶ cells/ml.

17. The method according to claim 11, wherein said at least first cell population comprises NK lymphocytes and/or T lymphocytes.
